(19) United States
(12) Patent Application Publication
(10) Pub. No.: US 2004/0236372 A1
(43) Pub. Date: Nov. 25, 2004

(54) Suture clamp

(76) Inventors: William E. Anspach III, Stuart, FL (US); Eddy Del Rio, Royal Palm Beach, FL (US)

Correspondence Address: Norman Friedland, Suite 400, 11300 US Highway One, North Palm Beach, FL, 33408, US

(21) Appl. No.: 10/441,834

(22) Filed: May 20, 2003

(51) Int. Cl. A61B 17/04

(52) U.S. Cl. 606/232

(57) ABSTRACT

A clamp for securing suture made from a main body that is generally a planar member that is configured into a generally circular, oval, or triangular shape or the like, with the top edges located in juxtaposed position to define a gap that is smaller in width than the diameter of the suture. A slot formed on the bottom of the main body is sufficiently large to allow the passage of four strands of the suture to pass there through and defines a double loop around each side edge of the main body to uniformly absorb the loads imposed on the suture. The method includes threading the suture through the slot and then looping it back into the slot, drawing the suture to the side edges so that the surgeon can tighten the suture or slide the clamp to a desired position and then locking the suture by sliding each strand into the gap which may be serrated.

### CROSS REFERENCES

This invention relates to the co-pending patent application entitled SURGICAL METHOD FOR SUTURING TENDONS/LIGAMENTS TO BONES filed by WILLIAM E. ANSPACH III, the co-inventor of this application and filed contemporaneously and is commonly assigned to The Anspach Effort, Inc. and identified by Attorney Docket No. N1053 and is incorporated herein in its entirety by reference.

### TECHNICAL FIELD

This invention relates to surgical devices that are utilized to secure sutures and more particularly to a clamp that includes judicious passages that permit the judicious threading of the suture in such a way as to provide positive securing thereof A double loop configuration of the suture inserted into the clamp relieves the strain adjacent the gripping surface of the clamp to enhance the life of the suture and improve the holding capability of the tying.

### BACKGROUND OF THE INVENTION

As is well known in the medical technology, surgeons have sundry method of tying sutures together with knots and clamps. However, it is known that heretofore methods of tying sutures with knots and/or clamps are not satisfactory for many types of medical repairs. Also, it is very difficult for the surgeon to tie the sutures in an arthroscopic type of surgery because of the amount of space, or actually the lack of space, needed to manipulate the sutures into a knot. The heretofore known clamping devices are either too complicated to use or do not have the desired securing properties to hold the sutures in place when subjected to heavy and/or repeated loads. For example, the tension that occurs when a torn tendon is tied to the bone structure of the patient, the movement of the patient imparts a load on the suture tending to pull the suture away from the clamp.

We have found that we can obviate this problem by providing a uniquely designed clamp that is threaded outside the wound and includes loops of two strands of the suture within the clamp so as to more uniformly take up the load that is transmitted by the tension that the patient creates by virtue of body movement. The design of the clamp includes a unique structure that in combination with the double looping imparts a strong holding force on the suture. Once the suture is threaded, the surgeon can then slide the clamp into the wound until it reaches its target.

### SUMMARY OF THE INVENTION

The object of this invention is to provide for sutures an improved clamp.

A feature of this invention is a clamp that is formed from a planar member that is bent so that the edges are juxtaposed and defines a space for receiving the suture and that is smaller in the transverse direction than the diameter of the suture and may be serrated to define the gripping surface and is angled so that the clamp can be moved in only one direction when fully threaded.

A further feature of this invention is the inclusion of a slot on the bottom of the clamp diametrically opposed to the top space and is sufficiently long to provide passages for up to four strands of the suture.

A still further object of this invention is to provide radii on the clamp at each juncture where the suture abuts against the surfaces of the clamp.

A further object of this invention is to provide another embodiment of this invention where the cross sectional shape of the clamp is triangular.

Another object of this inventions is the method for threading a clamp with suture so as to secure and retain the suture. The threading of the clamp occurs outside the wound of the patient and is drawn into the wound until it reaches its target and cannot be moved in the opposite direction without dismantling the suture.

This invention is characterized as being simple to make, relatively inexpensive, ease of use and enhances the life of the suture being utilized in a tying procedure.

The foregoing and other features of the present invention will become more apparent from the following description and accompanying drawings.

### BRIEF DESCRIPTION OF THE DRAWINGS

FIGS. 1a-1d illustrate the method of threading the clamp of this invention with suture for securing and retain the suture;

FIG. 2 is a perspective view of this invention illustrating the invention being utilized for securing and retain the suture;

FIG. 3 exemplifies another embodiment of this invention where the cross-sectional view is triangularly shaped; and

FIG. 4 is a sectional view taken through lines 4-4 of FIG. 3.

These figures merely serve to further clarify and illustrate the present invention and are not intended to limit the scope thereof.

### DETAILED DESCRIPTION OF THE INVENTION

This invention is shown as an example for tying tissue to bone in co-pending patent application entitled SURGICAL METHOD FOR SUTURING TENDONS/LIGAMENTS TO BONES, supra, and has sundry applications as will be described hereinbelow. While this invention can be utilized for fixing torn tissue by tying the tissue to bones by surgeons performing general or arthrosporic surgery, as will be understood by those skilled in this technology, this invention is not so limited and can be utilized wherever it is necessary to secure sutures. Suffice it to say that this invention has applicability in any medical procedure where some type of tying needs to be performed and a clamp is acceptable in that procedure.

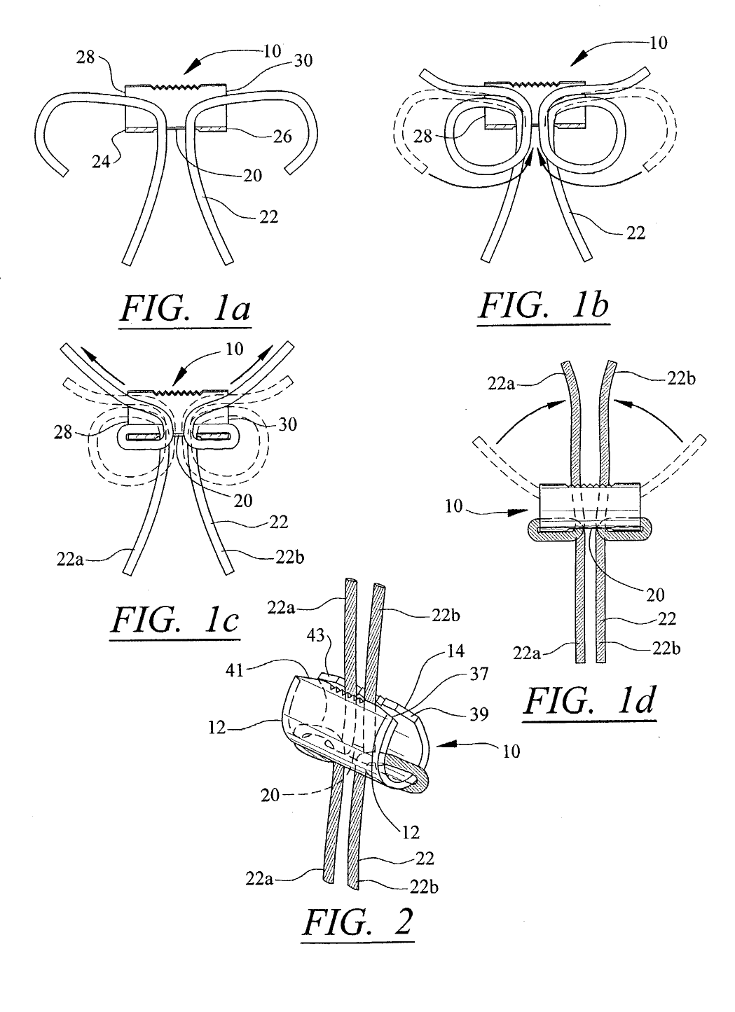

As best seen in FIGS. 1a-1d and 2, the clamp generally illustrated by reference numeral 10 is configured in a circular or oval shape by folding a planar shaped flat main body 12 so that the edges 14 and 16 are in juxtaposition and define the straight through gap 18. Slot 20 is formed at the bottom of clamp 10 and is diametrically opposed to gap 18 and extends a sufficient axial length to allow the passage of at least four strands of the suture 22. The method of threading the suture in the clamp so as to secure and retain the same is by passing each strand 22a and 22b of suture 22 into slot 20 and looping each strand around the edges 24 and 26 of ends 28 and 30 respectively, and then returning the strands through the slot 20 and directing each strand to the ends 28 and 30 of clamp 10. Typically, the surgeon will thread the suture to the clamp outside the wound and once threaded he can then pull the clamp to slide down the strands of suture until it reaches its target. In operation and as mentioned above, the surgeon threads the clamp to obtain the double loop configuration and from without the ends of the clamp draws the strands into the clamp and up through the gap 18. The width of the gap 14 is dimensioned so that it is narrower than the diameter of the suture and the edges 34 and 36 are serrated so as to bind into the braid of the suture. Further, the edges 34 and 36 are angularly disposed relative to the centerline of the clamp so that it defines a negative rake. This negative rake allows either the strands to be moved in one direction and not the other or the clamp can be moved in one direction and not the other. Hence, once the surgeon places the suture into the gap of the clamp, the suture becomes secure and is then retained thereby and the surgeon can then slide the clamp from outside the wound, through the wound until it reaches its destination at the target. The ends 37, 39, 41 and 43 may be beveled to facilitate the entry of the suture when the surgeon is locking the suture to the clamp.

Any tension or pulling of the bottom portion of the suture by the body portion being secured thereby will be absorbed by the bottom of the clamp 10 and the force will be distributed evenly across the bottom thereof where the suture is double looped. This looping or, rather, double looping configuration improves the holding characteristics of the clamp and at the same time prevents or at least improves the possibility of the suture from rupturing or breaking since the load is removed from the gripping portion (serrated edges) of the clamp.

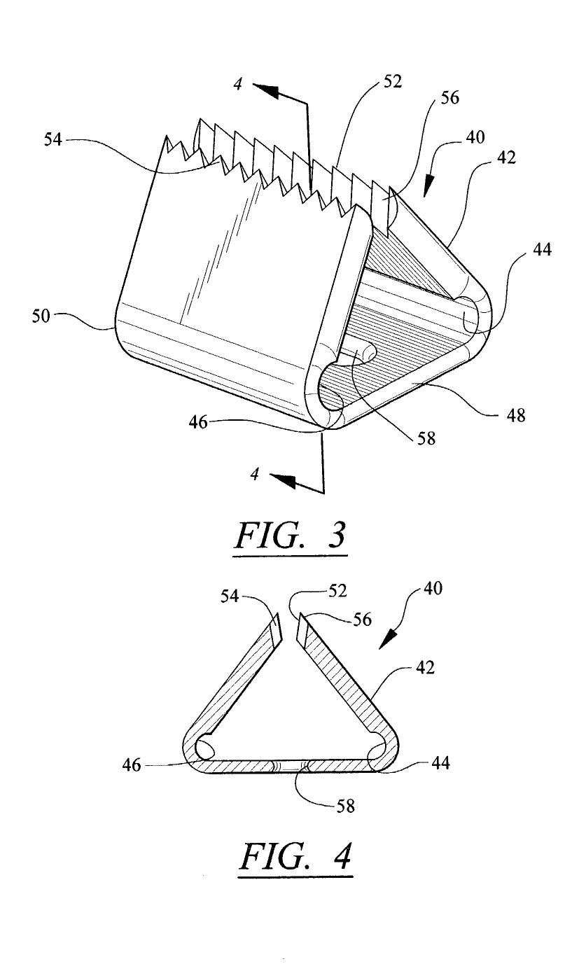

FIGS. 3 and 4 exemplify another embodiment of this invention where the clamp 40 is triangular shaped in cross section. As can be seen in the FIGS. 3 and 4, the clamp 40 is made similarly to the clamp depicted in FIG. 2 and is formed from a planar rectangularly shaped main body 42 that is bent into the configuration as shown. The inner portion of the main body 42 is milled out at the corners 44 and 46 to form a reduced diameter portion extending from the front edge 48 to the rear edge 50 for facilitating the bending of the planar body 42 and forming a uniform symmetrical member. The top edges 52 and 54 are juxtaposed and are spaced to define the gap 56 which may be serrated as shown. The slot 58 is located diametrically opposite the gap 56 defined by the top edges 52 and 54. The edges of slot 58 are rounded as are the fore and aft edges 48 and 50 so as to allow the ease of movement of the clamp and to prevent fraying of the braided portion of the suture. As is the case of the clamp depicted in FIG. 2, the width of the slot 56 is narrower than the diameter of the suture, say in the order of 4 to 1, so as to clasp the suture and hold it firmly in place. The threading of the suture to the clamp is identical for both embodiments.

In operation, the surgeon will install the clamp to the suture outside the wound of the patient by first inserting both strands of the suture through the opening formed by the aperture 20 located at the bottom of the clamp. The surgeon will then loop each strand around the bottom of the clamp and thread each strand to reenter the aperture 20. The ends of the two strands are then positioned outside the side edges of the clamp and then inserted into the gap 14 until each of the two strands are drawn between the serrated edges. Since these edges are angularly disposed in such a direction to define a negative rake, the strands can be drawn in the upward direction as viewed in FIG. 2 but not in the downward direction. Obviously, the clamp can likewise be moved but only in the downward direction. When the clamp is threaded as shown in FIG. 2, the surgeon can then position the clamp from outside the wound to within the wound until it reaches its destination at the target.

What has been shown by this invention is a simple clamp that double loops the suture at the bottom so as to absorb the load and remove the load from the serrated edges so as to enhance the carrying capacity of the suture and increase its life by prevention the suture from breaking or at the very least improving the breaking characteristics of the tied suture.

Although this invention has been shown and described with respect to detailed embodiments thereof, it will be appreciated and understood by those skilled in the art that various changes in form and detail thereof may be made without departing from the spirit and scope of the claimed invention.

It is claimed:

1. A clamp for a suture including a generally planar main body configured into a generally rounded shape in cross section having the top edges in juxtaposition relative to each other for defining a straight through gap whose width is dimensioned smaller than the diameter of the suture, and a slot located at the bottom of the main body in opposition to said gap and dimensioned to allow the passage of up to four strands of the suture there through.

2. A clamp for a suture as claimed in claim 1 on wherein the edges of said slot are rounded.

3. A clamp for a suture as claimed in claim 2 wherein the side edges of said main body in proximity to said slot are rounded.

4. A clamp for a suture including a generally planar main body configured into a generally triangular shape in cross section having the top edges in juxtaposition relative to each other for defining a straight through gap whose width is dimensioned smaller than the diameter of the suture, and a slot located at the bottom of the main body in opposition to said gap and dimensioned to allow the passage of up to four strands of the suture there through.

5. A clamp for a suture wherein said planar member having a fore edge and an aft edge and including a pair of spaced milled out portions extending from the fore edge to the aft edge and each of the space milled out portions being spaced axially relative to each other and located at the bends of the triangular shape of the main body.

6. A clamp for a suture as claimed in claim 5 wherein the edges of the slot are rounded and the edge portion of said fore edge and said aft edge adjacent to said slot is rounded.

7. The method of threading suture to a clamp where the clamp includes a main body that has a straight through gap at the top end and a slot on the bottom in opposition to the gap and the width of the gap is smaller than the diameter of the suture and the slot is capable of passing four strands of the suture there through including the steps of:
outside of the wound of the patient,
i) threading one end of the suture through the slot and folding it over the side edge of the main body adjacent thereto and looping that end into the slot and positioning the end thereof toward the side edges of the main body;
ii) threading the other end of the suture through the same slot of step i) and folding it over the side edge of the main body adjacent thereto and looping that end into the slot and positioning the ends thereof toward the side edges of the main body;
iii) sliding each of the ends into the gap whose dimension is smaller than the diameter of the suture; and
iv) sliding the clamp down the suture from outside the wound and into the wound until it reaches the target of the medical procedure.
